(19) United States
(12) Patent Application Publication
(10) Pub. No.: US 2009/0244481 A1
(43) Pub. Date: Oct. 1, 2009

(54) VISION RECOVERY TRAINING DEVICE

(75) Inventors: Toshiharu Satoh, Tokyo (JP); Eiichi Ookawa, Kanagawa (JP); Hidenori Horie, Kanagawa (JP)

Correspondence Address: PEARNE & GORDON LLP, 1801 EAST 9TH STREET, SUITE 1200, CLEVELAND, OH, 44114-3108, US

(73) Assignees: Matsushita Electric Industrial Co., Ltd., Osaka (JP); TechnoMaster Co., Ltd., Kanagawa (JP)

(21) Appl. No.: 12/088,042

(22) PCT Filed: Sep. 29, 2006
(86) PCT No.: PCT/JP2006/319580

§ 371 Date: Mar. 25, 2008

(30) Foreign Application Priority Data
Sep. 30, 2005 (JP) 2005-288552
Sep. 30, 2005 (JP) 2005-288553

(51) Int. Cl. A61B 3/00

(52) U.S. Cl. 351/203

(57) ABSTRACT

It is an object of the present invention to provide a vision recovery training device that can more improve a training effect of a trainer whose visual acuity is lowered.

The vision recovery training device of the present invention trains an image formation accommodative function of an eye. The vision recovery training device includes: an eyepiece 3 that allows the trainer to watch a visual acuity training image in a vision recovery training device main body; and a distance maintaining part 14 that maintains a distance between the trainer and the eyepiece 3.

### TECHNICAL FIELD

The present invention relates to a vision recovery training device for training an image formation accommodative function of an eye.

### BACKGROUND ART

Ordinarily, the decrease in a visual acuity is considered to be caused from the decrease in the image formation accommodative function of the eye due to the strain or collapse of an accommodation of an accommodative muscle of an eye (a ciliary muscle). Thus, this muscle is trained so that the recovery of the visual acuity can be anticipated. Usually, a method has been carried out that the ciliary muscle has been activated by training to activate an accommodation function and thus improve the visual acuity.

For instance, a vision recovery training device is proposed that enhances an effect of improving the visual acuity by activating the work of the above-described accommodative muscle (for instance, see Patent Document 1). In this vision recovery training device, a watching plate on which a mark is displayed is reciprocated to allow the mark to come closely to or to separate from a trainer. When the trainer of the device watches carefully this mark, since the focus of an eye continuously moves between a near distance and a remote distance, the accommodative muscle is efficiently trained.

In the above-described vision recovery training device, when the trainer does not gaze at the mark, an effect of the training cannot be obtained. Accordingly, various kinds of contrivances are made. For instance, an entire part of the device is accommodated in a case, so that even the watching plate on which the mark is displayed is reciprocated in a cavity, a disturbance is reduced to easily gaze at the mark.

### Patent Document 1: JP-A-6-339501

### DISCLOSURE OF THE INVENTION

### Problems that the Invention is to Solve

Since the trainer who uses the vision recovery training device has the visual acuity lowered, the trainer ordinarily remedies the visual acuity by glasses and the like. When the trainer does not remedy the visual acuity to carry out a training with the naked eye, the trainer whose visual acuity is extraordinarily lowered hardly visually recognize the mark itself. Further, when the trainer wears glasses to train eyes to ensure his or her visual acuity, if the trainer peeps into the case in which the entire part of the device is accommodated, a situation may possibly arise that the glasses come into contact with the case, and accordingly, the glasses rather disturb the trainer and the trainer cannot concentrate on the training. Further, a trainer who wears contact lenses to remedy a visual acuity desires to avoid the eye from being attached to an eyepiece and pressed thereby.

The present invention is devised by considering the above-described circumstances and it is an object of the present invention to provide a vision recovery training device that can more improve a training effect of a trainer whose visual acuity is lowered.

### Means for Solving the Problems

A vision recovery training device of the present invention concerns a vision recovery training device for training an image formation accommodative function of an eye. The vision recovery training device includes: an eyepiece that allows a trainer to watch a visual acuity training image in a vision recovery training device main body; and a distance maintaining part that maintains a distance between the trainer and the eyepiece.

According to the above-described configuration, since the trainer does not need to mind the distance to the eyepiece and easily concentrates a consciousness on the visual acuity training image, the trainer can efficiently carry out a visual acuity recovery training.

Further, in the vision recovery training device of the present invention, the distance maintaining part allows the forehead of the trainer to abut on so as to maintain the distance between the trainer and the eyepiece.

According to the above-described configuration, the movement of the head of the trainer can be suppressed, so that the line of vision can be stabilized during a training and a visual acuity recovery training operation can be efficiently carried out.

Further, in the vision recovery training device of the present invention, the distance maintaining part allows the forehead and jaw of the trainer to abut on so as to maintain the distance between the trainer and the eyepiece.

According to the above-described configuration, the movement of the head of the trainer can be suppressed, so that the line of vision can be stabilized during a training and a visual acuity recovery training operation can be efficiently carried out.

Further, in the vision recovery training device of the present invention, the distance maintaining part maintains the distance between the trainer and eyepiece to a distance in which glasses worn by the trainer do not contact the eyepiece.

According to the above-described configuration, since there is no troublesomeness that the trainer wearing the glasses allows the glasses to collide with the eyepiece and thus the trainer can concentrate a consciousness on the training, the trainer can efficiently carry out the visual acuity recovery training.

The vision recovery training device of the present invention further includes: a visual acuity remedying part that remedies the visual acuity of the trainer to be suitable for a training.

According to the above-described configuration, since the trainer can concentrate a consciousness on the training under state that the trainer maintains a suitable visual acuity, a training effect to the trainer whose visual acuity is lowered can be more improved.

Further, in the vision recovery training device of the present invention, the visual acuity remedying part remedies the visual acuity of the trainer to such a visual acuity as to catch a moving mark.

According to the above-described configuration, even when the trainer has a myopia or a presbyopia, since the visual acuity of the trainer is remedied during the training to a suitable visual acuity in which the trainer does not feel tired, a training effect can be improved.

Further, in the vision recovery training device of the present invention, the visual acuity remedying part has a structure that a visual acuity remedying lens is attached to the eyepiece.

According to the above-described configuration, since the trainer simply peeps at the eyepiece to remedy the visual acuity to a proper visual acuity, the trainer is not annoyed and can concentrate on the training. Accordingly, the trainer can efficiently carry out the visual acuity recovery training without lowering a concentration of the trainer.

Further, the vision recovery training device of the present invention further includes: a watching part that displays the visual acuity training image in a housing; and a display control part that changes the display size of the visual acuity training image so that the visual acuity training image is projected on the retina of the trainer with a definite size in accordance with an optical distance between the position of the eye of the trainer and the position of the watching part.

According to the above-described configuration, since the size of a figure whose image is formed on the retina is fixed, an accommodative muscle can be effectively exercised.

### ADVANTAGE OF THE INVENTION

According to the present invention, a training effect to the trainer whose visual acuity is lowered can be improved.

### BRIEF DESCRIPTION OF THE DRAWINGS

FIG. 1 is a partly broken perspective view showing the structures of main parts of a vision recovery training device of a first embodiment of the present invention.

FIG. 2 is a cross-sectional view showing a schematic structure of the vision recovery training device of the first embodiment of the present invention.

FIG. 3 is a block diagram showing a schematic functional structure of a controller for controlling the operation of the vision recovery training device of the first embodiment of the present invention.

FIG. 4 is a diagram showing examples of a mark displayed when a watching part of the vision recovery training device of the first embodiment of the present invention is moved.

FIG. 5 is a cross-sectional view showing a schematic structure of a vision recovery training device of a second embodiment of the present invention.

FIG. 6 is an explanatory view showing a state that a trainer wearing glasses carries out a training in the second embodiment of the present invention.

FIG. 7 is an explanatory view showing a state that a trainer wearing glasses carries out a training in the second embodiment of the present invention.

FIG. 8 is a diagram showing a relation between a distance from the eyepiece of the watching part and the brightness of the mark.

FIG. 9 is a cross-sectional view showing a structure in which a display unit is provided separately from a watching part.

### DESCRIPTION OF REFERENCE NUMERALS AND SIGNS

1 inner space of housing
2 storing part of watching part moving unit
3 eyepiece
4 watching part
5 display part
6 mark
7 visual acuity remedying part
8 brace
9 pedestal
10 pulley
11 pulley
12 belt
13 motor
14 distance maintaining part
15 distance maintaining part
16 display unit
20 controller
21 main control part
22 watching part movement control part
23 watching part display controller
24 operating part

### BEST MODE FOR CARRYING OUT THE INVENTION

Now, a vision recovery training device of embodiments of the present invention will be described below by referring to the drawings.

### First Embodiment

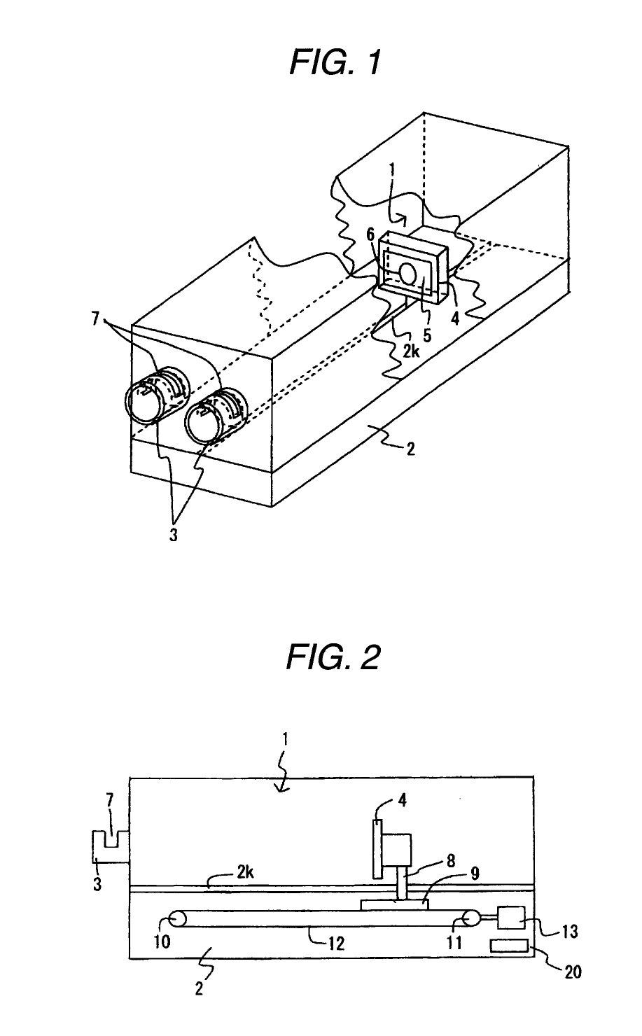

FIG. 1 is a partly broken perspective view of the structures of main parts of the vision recovery training device of a first embodiment of the present invention. This device roughly includes two components of an inner space 1 of a housing and a storing part 2 of a watching part moving unit. Further, this device is constructed so that a trainer can train a visual acuity without receiving a disturbance except a watching part 4 in the housing arranged at a position where the trainer can visually watch from an eyepiece 3, for instance, the watching part moving unit or a landscape in the periphery of the device.

The eyepiece 3 is provided on one of wall surfaces of the inner space 1 of the housing. The trainer allows an eye to abut on the eyepiece 3 so that the trainer can watch a mark 6 as a visual acuity training image displayed on a display part 5 of the watching part 4 in a vision recovery training device main body. At least one eyepiece 3 is provided and two eyepieces 3 are preferably provided. A visual acuity remedying part 7 serves to remedy the visual acuity of the trainer to a visual acuity suitable for a training and is formed by attaching a visual acuity remedying lens that is not shown in the drawing to a groove formed in the eyepiece 3.

In this embodiment, the watching part 4 has the display part 5 as an electronic display unit such as a display by a light emitting element, for instance, a CRT (Cathode Ray Tube), a liquid crystal display, an organic EL (Electro-Luminescence), etc. so that the mark to be watched by the trainer can be suitably changed. The watching part is reciprocated by the watching part moving unit accommodated in the storing part 2 of the watching part moving unit to change a distance to the eyepiece 3.

FIG. 2 is a cross-sectional view showing a schematic structure of the vision recovery training device of this embodiment. The watching part 4 is fixed to a pedestal 9 by a brace 8 or other suitable unit. The pedestal 9 is mounted on a conveying unit formed by pulleys 10 and 11, a belt 12 or the like. Further, the above-described watching part moving unit forms a belt conveyor by, for instance, the pulleys 10 and 11 and the belt 12. The belt 12 is connected to the watching part 4 and the pulley 11 is driven by changing a rotating direction by, for instance, a motor 13 to reciprocate the watching part 4. Further, a nut of a ball screw is connected to the watching part 4 and the ball screw is rotated by changing a rotating direction by, for instance, the motor 13 so that the reciprocating movement of the watching part 4 can be realized.

In a boundary of the inner space 1 of the housing and the storing part 2 of the watching part moving unit, an opening part 2k is provided that has a requisite minimum size necessary for connecting the watching part 4 to the watching part moving unit to reciprocate the watching part 4. Further, the operation of the motor 13 and the display of the watching part 4 are controlled by a below-described controller 20. Though not shown in the drawing, the controller 20 is connected to the display part 5 and the motor 13 by a signal line for transmitting and receiving a control signal.

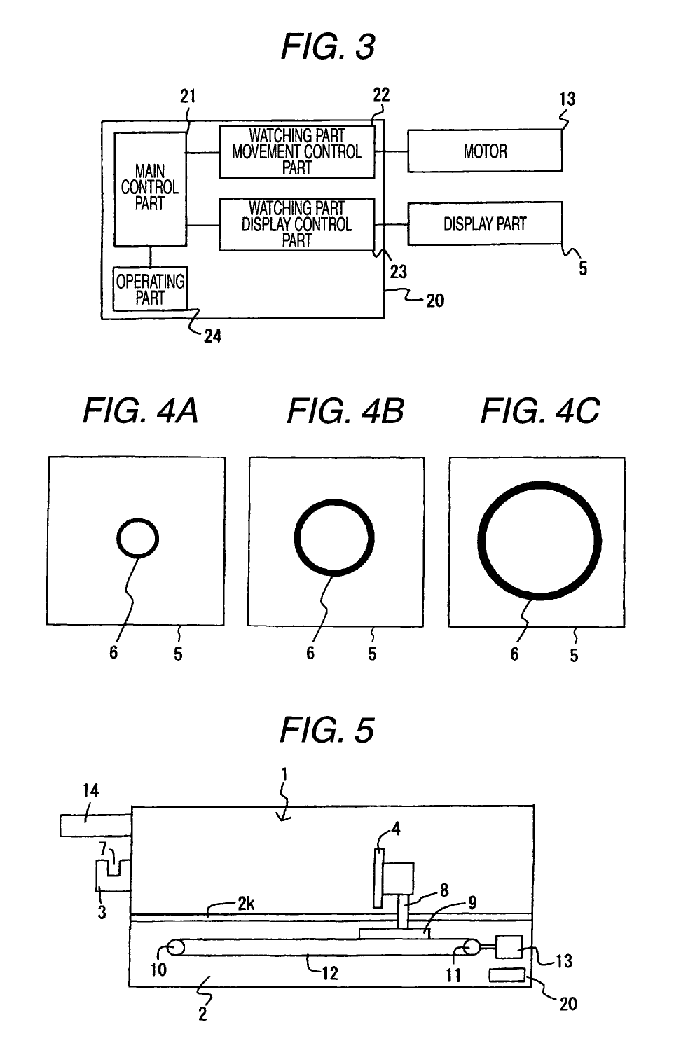

FIG. 3 is a block diagram showing a functional structure of the controller 20 for controlling the operation of the vision recovery training device of this embodiment. The controller 20 includes a main control part 21, a watching part movement control part 22 for controlling the driving of the motor 13, a watching part display control part 23 for controlling the display of the display part 5, and an operating part 24.

The main control part 21 controls the operation of the entire part of the vision recovery training device to control the watching part movement control part 22 and the watching part display control part 23 in accordance with an instruction of the operating part 24. The watching part movement control part 22 outputs a driving signal to the motor 13 to control the movement of the watching part 4. At this time, the watching part movement control part obtains information corresponding to the setting of the motor 24 from the main control part 21 to control a moving range and a moving speed of the watching part 4. The watching part 4 may be continuously or intermittently moved. The watching part display control part 23 drives the display part 5 composed of the electronic display unit such as a display by the light emitting element, for instance, the CRT, the liquid crystal display, the organic EL, etc. to display the mark 6 to be watched by the trainer.

Further, the watching part display control part 23 changes the display size of the mark so that the mark 6 of the watching part 4 is projected on the retina of the trainer with a definite size in accordance with an optical distance between the position of an eye of the trainer and the position of the watching part 4. Information of the optical distance between the eyepiece 3 and the watching part 4 is obtained from the main control part 20. Data for displaying the mark 6 proportional to the optical distance between the eyepiece 3 and the watching part 4 may be calculated for each time, or information previously stored in a storing unit (not shown in the drawing) for storing operating information may be read and displayed. At that time, as the mark 6 to be displayed, a mark previously stored in the storing unit may be selected and designated by the operating part 24 every time the mark is required, or the mark 6 desired by the trainer may be designated and used from the operating part 24. In this case, data for displaying the mark 6 having a size proportional to the optical distance between the eyepiece 3 and the watching part 4 needs to be calculated for each time. As for the mark 6, any of marks such as a character, a figure, a symbol, and an image, etc. may be used at which the trainer easily looks and focuses his or her eye on.

When a visual acuity recovery training is performed by using the vision recovery training device constructed as described above, the trainer peeps into the inner space 1 of the housing from the eyepiece 3 to watch the mark 6 on the watching part 4. Since the position of the mark 6 is changed by the movement of the watching part 4, the trainer watches the mark 6 located at different positions. Accordingly, the eye needs to be focused correspondingly to the change of the position of the mark 6 so that an image formation accommodative function of the eye such as a ciliary muscle is operated. Thus, the enhancement of the image formation accommodative function of the eye can be anticipated. At this time, the optical distance between the eye of the trainer and the mark 6 is preferably changed between a near point distance and a far point distance of a person having a normal visual acuity.

FIG. 4 is a diagram showing one example of the mark 6 displayed when the watching part 4 of the vision recovery training device of this embodiment is moved. FIG. 4A shows a display example of the mark 6 at a position nearest to the trainer, FIG. 4B shows a display example of the mark at an intermediate position between the nearest position and the remotest position and FIG. 4C shows a display example of the mark at the remotest position, respectively. In such a way, the marks 6 of mutually similar forms are displayed with sizes proportional to the distance from the trainer. Thus, even when the distance changes, since the size and the form of the mark 6 seen from the trainer do not substantially change, a quantity of information inputted to the brain of the trainer does not change and the trainer can easily focus his or her eye on the mark.

Here, the trainer using the vision recovery training device ordinarily has a lowered visual acuity. When the visual acuity is extraordinarily lowered, the trainer may hardly visually recognize the mark 6 with the naked eye, so that a training effect may be possibly deteriorated. That is, a suitable visual acuity needs to be ensured in recovery training the visual acuity to improve the training effect. Thus, in the vision recovery training device of this embodiment, as the visual acuity remedying part 7, a groove for attaching a visual acuity remedying lens is provided in the eyepiece 3. Thus, the suitable visual acuity can be ensured by attaching the visual acuity remedying lens suitable for the trainer before a training is started. The visual acuity suitable for the training is such a visual acuity as to see clearly the mark from a near point to a far point. The visual acuity remedying part 7 is adequate enough to remedy the visual acuity of the trainer so as to catch the moving mark. Since the excessive remedy of the visual acuity causes the training effect to be reduced and a tiredness to arise, the visual acuity needs to be avoided from being excessively remedied.

Since a mark ordinarily used in the measurement of the visual acuity such as a Landolt ring or a Snellen chart is employed as the mark 6 used when the visual acuity remedying lens is selected beforehand, a quantitative visual acuity can be gained. The visual acuity remedying lens may be selected so that the trainer has a vision in which the trainer can visually recognize the mark both in the near point and the far point while the size of the mark 6 is changed so as to test the visual acuity in all distances and a load is not applied to the trainer.

As described above, in the vision recovery training device of this embodiment, when the visual acuity recovery training is performed, the trainer simply peeps at the mark 6 from the eyepiece 3, so that the trainer can see the mark 6 with the visual acuity suitable for the training. Accordingly, under a state that the trainer maintains the suitable visual acuity, the trainer can concentrate his or her consciousness on the training. Therefore, the training effect can be more improved to the trainer whose visual acuity is lowered and a visual acuity recovery training operation can be efficiently realized.

### Second Embodiment

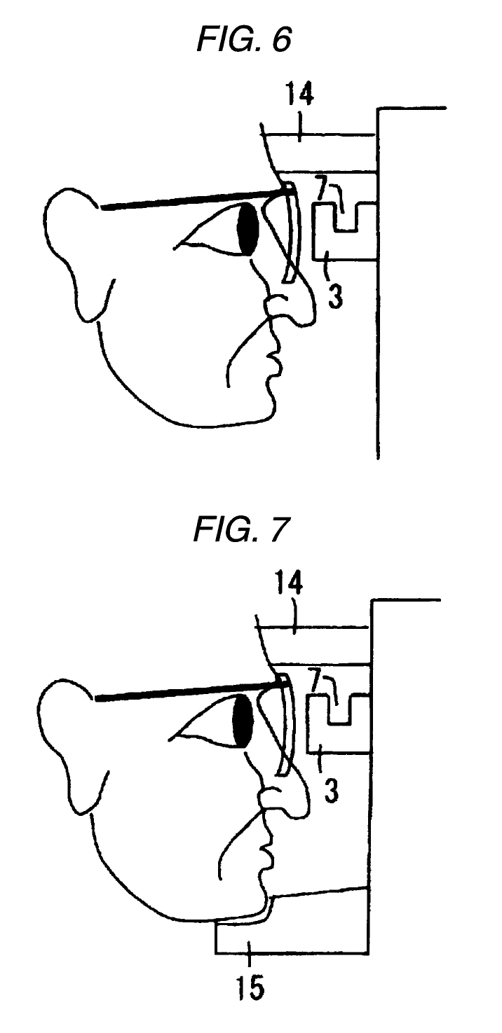

FIG. 5 is a cross-sectional view showing a schematic structure of a vision recovery training device of a second embodiment of the present invention. A difference of the second embodiment from the first embodiment shown in FIG. 2 resides in that a distance maintaining part 14 for ensuring a distance between the face of a trainer and an eyepiece 3 is provided on an upper part of the eyepiece 3. In this embodiment, the vision recovery training device having a visual acuity remedying part 7 and the distance maintaining part 14 is shown, however, when the distance maintaining part 14 is provided, the visual acuity remedying part 7 is not essentially necessary. When a visual acuity recovery training is performed by using the vision recovery training device of this embodiment, the trainer peeps into an inner space 1 of a housing from the eyepiece 3 like the first embodiment to watch a mark 6 on a watching part 4. At this time, under a state that the trainer allows a forehead to abut on the distance maintaining part 14, the trainer peeps at the eyepiece 3. An amount of protrusion of the distance maintaining part 14 from the housing of the device can be adjusted to adjust a distance to the trainer. The trainer who allows his or her forehead abut on the distance maintaining part 14 to maintain a suitable distance from the eyepiece 3 does not have an eye compressed by the eyepiece 3. Especially, the trainer who wears a contact lens can train the visual acuity under a preferable state. Further, since the forehead abuts on the distance maintaining part 14, the movement of a head is suppressed. When the head does not move, since the line of vision during a training is stabilized so that the trainer can easily watch the mark 6 on the watching part 4, a training effect is more improved.

FIG. 6 is a diagram showing a state that the trainer wearing glasses carries out a training of a visual acuity in this embodiment. When the trainer wearing the glasses carries out the vision recovery training by the vision recovery training device of this embodiment, if the trainer has a remedied visual acuity suitable for the training, the glasses ordinarily used by the trainer are used to carry out the training. At this time, there is a possibility that the glasses collide with the eyepiece 3. When the glasses actually collide with the eyepiece, the trainer feels uncomfortable, so that a concentration to the training is interrupted. Further, a situation that the trainer carries out the training of the visual acuity under a state that the trainer pays attention not to allow the glasses to collide with the eyepiece 3 is troublesome for the trainer and causes the concentration to be deteriorated. In the vision recovery training device of this embodiment, an amount of protrusion of the distance maintaining part 14 can be adjusted and the amount of protrusion of the distance maintaining part 14 can be previously adjusted so that a distance is maintained in which the glasses do not collide with the eyepiece 3 under a state that the trainer wears the glasses. Under this state, when the vision recovery training is performed, the trainer can suppress the movement of a head. Furthermore, since the glasses do not collide with the eyepiece 3, the trainer can concentrate on a visual acuity recovery training operation while the trainer wears the glasses. Thus, a training effect can be more improved.

As described above, in this embodiment, since the distance maintaining part 14 is provided for ensuring the distance between trainer and the eyepiece 3, the trainer can concentrate his or her consciousness on the training under a state that a suitable visual acuity ensured so that the effect of the training is more improved to the trainer whose visual acuity is lowered, and the visual acuity recovery training can be efficiently carried out.

In the configuration shown in FIGS. 5 and 6, the distance maintaining part 14 is provided that abuts on the forehead of the trainer. However, as shown in FIG. 7, a distance maintaining part 15 may be further provided that abuts on a jaw part of the trainer. Thus, an effect can be more exhibited that the distance between the face of the trainer and the eyepiece 3 is maintained and the movement of the head of the trainer is suppressed.

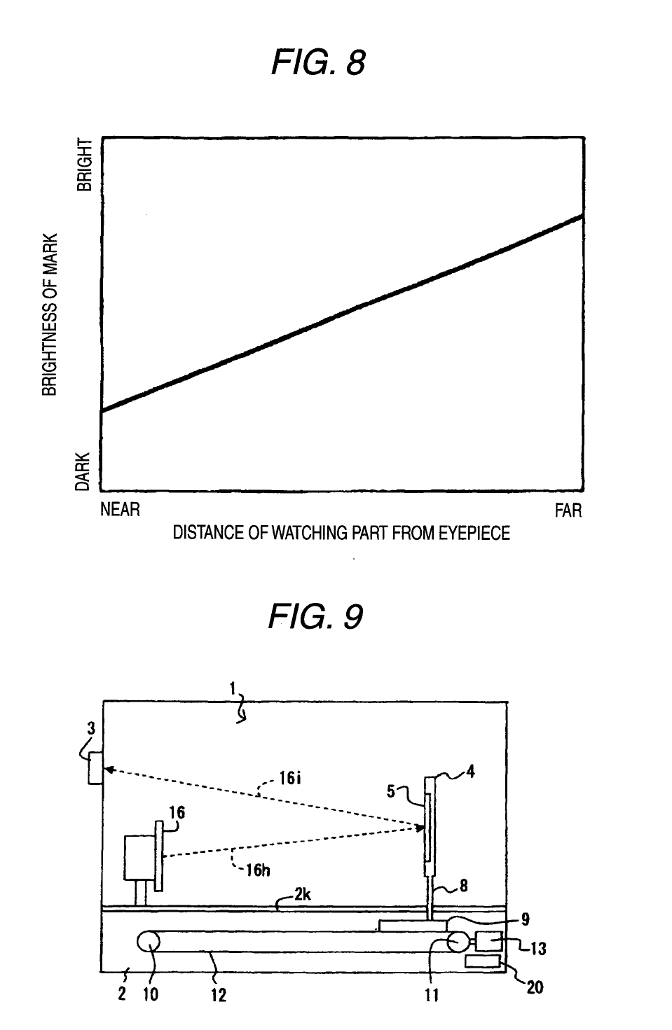

The vision recovery training device of the embodiments of the present invention is described above. As shown in FIG. 8, as the distance from the eyepiece 3 becomes greater, the brightness of the mark 6 of the watching part 4 preferably becomes higher. Thus, as shown in FIG. 9, a display unit 16 for displaying the mark on a display part 5 of the watching part 4 may be provided separately from the watching part 4. According to this configuration, since the watching part can be moved and the display unit can be fixed, the brightness of the mark 6 can be more advantageously changed than a case that the movable display unit whose size or weight is limited. A sign 16h in the drawing designates a light of an image to the display part 5 from the display unit 16 and a sign 16i designates a reflected light to the eyepiece 3, respectively.

The present invention is explained in detail by referring to specific embodiments, and it is to be understood to a person with ordinary skill in the art that various changes or modifications may be made without departing from the spirit and scope of the present invention.

This application is based on Japanese Patent Application (No. 2005-288552) filed on Sep. 30, 2005 and Japanese Patent Application (No. 2005-288553) filed on Sep. 30, 2005 and contents thereof are incorporated herein as references.

### INDUSTRIAL APPLICABILITY

The present invention can effectively more improve a training effect to the trainer whose visual acuity is lowered and is useful for a vision recovery training device for training the image formation accommodative function of an eye.

1. A vision recovery training device for training an image formation accommodative function of an eye, comprising:
an eyepiece that allows a trainer to watch a visual acuity training image in a vision recovery training device main body; and
a distance maintaining part that maintains a distance between the trainer and the eyepiece,
wherein the distance maintaining part allows the forehead of the trainer to abut on so as to maintain the distance between the trainer and the eyepiece.

2. (canceled)

3. The vision recovery training device according to claim 1, wherein the distance maintaining part allows the forehead and a jaw of the trainer to abut on so as to maintain the distance between the trainer and the eyepiece.

4. The vision recovery training device according to claim 1, wherein the distance maintaining part maintains the distance between the trainer and the eyepiece in which glasses worn by the trainer do not contact the eyepiece.

5. The vision recovery training device according to claim 1, further comprising:
a visual acuity remedying part that remedies the visual acuity of the trainer to be suitable for a training.

6. The vision recovery training device according to claim 5, wherein the visual acuity remedying part remedies the visual acuity of the trainer to such a visual acuity as to catch a moving mark.

7. The vision recovery training device according to claim 5, wherein the visual acuity remedying part has a structure that a visual acuity remedying lens is attached to the eyepiece.

8. The vision recovery training device according to claim 1, further comprising:
a watching part that displays the visual acuity training image in a housing; and
a display control part that changes a display size of the visual acuity training image so that the visual acuity training image is projected on a retina of the trainer with a definite size in accordance with an optical distance between a position of the eye of the trainer and a position of the watching part.
